SEQUENCE LISTING (6 sequences)

SEQ ID NO: 1 - protein, 9 aa, Human papillomavirus 16
ISEYRHYCY

SEQ ID NO: 2 - protein, 9 aa, Human papillomavirus 16
YMLDLQPET

SEQ ID NO: 3 - protein, 9 aa, Human papillomavirus 16
TTLEQQYNK

SEQ ID NO: 4 - protein, 9 aa, Human papillomavirus 16
RAHYNIVTF

SEQ ID NO: 5 - protein, 8 aa, Human papillomavirus 16
RPRKLPQL

SEQ ID NO: 6 - protein, 9 aa, Human papillomavirus 16
LQPETTDLY

(19) United States
(12) Patent Application Publication
(10) Pub. No.: US 2026/0240974 A1
(43) Pub. Date: Aug. 20, 2026

(54) A THERAPEUTIC HPV VACCINE BASED ON VALIDATED TARGET EPITOPES

(71) Applicant: Deutsches Krebsforschungszentrum Stiftung des öffentlichen Rechts, Heidelberg (DE)

(72) Inventors: Angelika RIEMER, Heidelberg (DE); Maria Brauer, Heidelberg (DE); Jonas FÖRSTER, Heidelberg (DE); Jonas BECKER, Heidelberg (DE)

(73) Assignee: Deutsches Krebsforschungszentrum Stiftung des öffentlichen Rechts, Heidelberg (DE)

(21) Appl. No.: 19/160,464

(22) PCT Filed: Mar. 1, 2024
(86) PCT No.: PCT/EP2024/055430

§ 371 Date: Aug. 27, 2025

(30) Foreign Application Priority Data
Mar. 2, 2023 (EP) 23159734.5

(51) Int. Cl. A61K 39/12; A61K 39/00; A61P 31/20; A61P 37/04

(52) U.S. Cl. CPC A61K 39/12; A61P 31/20; A61P 37/04; A61K 2039/55561; A61K 2039/60; A61K 2039/64; A61K 2039/70; A61K 2039/80; C12N 2710/20022; C12N 2710/20034

(57) ABSTRACT

The present invention relates to a vaccine against a human papillomavirus 16 (HPV16)-related virus providing at least six discrete immunization peptides consisting of the amino acid sequences of SEQ ID NOs:1 to 6, wherein said vaccine comprises a mixture of discrete peptides each comprising exactly one of said amino acid sequences; and to the vaccine for use in medicine and in eliciting an immune response in a human subject against HPV infection, as well as a kit related thereto.

The present invention relates to a vaccine against a human papillomavirus 16 (HPV16)-related virus providing at least six discrete immunization peptides consisting of the amino acid sequences of SEQ ID NOs:1 to 6, wherein said vaccine comprises a mixture of discrete peptides each comprising exactly one of said amino acid sequences; and to the vaccine for use in medicine and in eliciting an immune response in a human subject against HPV infection, as well as a kit related thereto.

At least 20% of human malignancies are caused by consequences of persistent infections. Cancers caused by infectious agents are attractive targets for cancer vaccination approaches, as they provide the opportunity to target antigens that are immunological non-self. Vaccination can be prophylactic, inducing an immune response that prevents infection in the first place, or therapeutic, stimulating the immune system into eradicating established disease. Prophylactic immunization against certain high-risk human papillomavirus (HPV) types or against hepatitis B has become the paradigm for cancer immunoprevention.

As for therapeutic vaccination, HPV-associated neoplasia could again emerge as the model case: induction and maintenance of the malignant phenotype depends on two viral oncoproteins, E6 and E7. It is known from studies on spontaneously regressing HPV-induced lesions, and from HPV prevalence in immunosuppressed and HIV-infected patients, that cell-mediated immune responses are crucial in clearing established HPV infection. Various forms of vaccines, including peptide vaccines, targeting T cells to E6 and E7 have already been explored.

Candidate peptides proposed for vaccination against HPV are abundant in the literature, e.g. from Bonsack et al. (2019), Cancer Immunol Res; 7(5):719; Blatnik (2018), Proteomics 18:1700390, DOI: 10.1002/pmic.201700390; Krishna et al. (2018) Cancer Res.2018; 78(21):6159; Tsang et al. (2017), Vaccine 35(19):2605; Kast et al., J Immunol 1994; 152(8):3904; Ressing et al., J Immunol 1995; 154(11):5934; Bourgault Villada et al., Clin Exp Immunol 2010; 159(1):45; Mizuuchi et al., Exp Mol Pathol 2012; 92(1):185); Hara et al., Int J Oncol 2005; 27(5):1371; Jang et al., Cancer 2012; 118(8):2173, and Riemer et al., J Biol Chem 2010; 285(38):29608. Regarding peptide vaccines, it had, however, to be realized over the years that the processes leading to an effective T cell response are complex and require more than potential binding of a peptide to an MHC. In the case of MHC class I presentation, a precursor peptide must be cleaved by the proteasome of a presenting cell to provide the peptide in the first place; moreover, T cell activation may require cross-presentation of the peptide on MHC class I by APCs, which may cleave precursor peptides differently from non-APC cells. Moreover, there must be a T cell with the propensity to recognize the presented peptide, which crucially depends on the reservoir of T cell receptors available (cf. e.g. Becker & Riemer (2022), Frontiers Immunol, doi: 10.3389/fimmu.2022.883989; Habib et al. (2022), Cells 6; 11(3):421. doi: 10.3390/cells11030421). Also, in cancer cells, immune evasion mechanisms may prevent proper presentation of HPV-derived peptides (Steinbach & Riemer (2018), Int J Cancer 142:224). In conclusion, experimental binding of a candidate peptide to MHC class I in itself, unfortunately, does not make this candidate peptide already a plausible candidate for a vaccine.

In view of the above, it is clear that verifying candidate HPV peptides as indeed suitable for vaccination is by far a non-routine task: in presentation assays, the amounts of peptide obtainable are minute, and for many peptides, fragmentation patterns in mass spectrometry are complex. It is, thus, that in a study of 2010 (Riemer et al. (2010), loc.cit.), of 21 peptides predicted to be presented via HLA-A*0201, only one could be confirmed to be presented. After eight years of method development, 17 peptides could be confirmed from a predicted group of 121 potential HLA-A2 binding peptides (Blatnik et al. (2018), loc. cit.). Notably, cysteine-containing peptides were excluded altogether, as is usual in the art, since they are prone to intra- and intermolecular reactions, which complicate analysis and further reduce the amounts of defined and detectable ions available for MS analysis. If a HPV peptide cannot be identified as suitable by the in vitro methods described above, the only remaining alternative is in vivo testing, which requires extended animal and/or human testing by vaccination.

Also, for successful therapeutic vaccination against HPV, it is not only necessary that the vaccinated peptide is immunogenic, e.g. by activating T cells when presented via MHC molecules, but the peptide also has to be presented by target cells, i.e. HPV-infected cells. As noted in Riemer et al. ((2010), loc. cit.), it is essential for vaccination to define HPV-16 E6 and E7 T cell epitopes that are naturally processed and presented on the surface of virally altered cells. Only those HPV peptide/MHC class I complexes are capable of being recognized by cytolytic T lymphocytes to target destruction of transformed cells. Thus, success of a therapeutic HPV vaccine is dependent on accurate identification of HPV epitopes displayed on HPV-infected cells. Apart from direct proof of MHC presentation by MS analysis, investigating memory responses in healthy donors can serve as a surrogate test for presentation. If memory immune responses are present, the epitope in question must have been presented during a previous encounter with the virus. However, even proven natural immunogenicity is not sufficient to validate a peptide in the context of its restricting HLA complex as an actionable target. This is because APCs use a different proteasome, the immunoproteasome, compared to normal body cells, and furthermore, antigen processing of HPV16-infected/transformed cells is heavily modulated by the virus. Thus, a memory T cell response only proves that the peptide was presented by antigen-presenting cells (APCs), not that this epitope will still be presented on HPV-dependent tumor cells. Thus, not every possible HPV epitope is presented on HPV-positive target cells.

Thus, to be an actionable target, a HPV16-derived peptide must bind to an HLA molecule, be immunogenic, and must also be presented on the target cell, in particular an HPV16 infected or transformed cell.

There is, thus, a need in the art for improved means and methods for immunoreactive agents against cells transformed by HPV, such as HPV-positive cancer cells, and reagents related thereto, without the drawbacks as referred to above.

The technical problem underlying the present invention can be seen as the provision of means and methods for complying with the aforementioned needs. The technical problem is solved by the embodiments characterized in the claims and herein below.

In accordance, the present invention relates to a vaccine against a human papillomavirus 16 (HPV16)-related virus providing at least six discrete immunization peptides consisting of the amino acid sequences of SEQ ID NOs:1 to 6, wherein said vaccine comprises a mixture of discrete peptides each comprising exactly one of said amino acid sequences.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof; thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified, in particular the polynucleotides and (poly)peptides, may be comprised in larger structures, e.g. may be covalently or non-covalently linked to further sequences, carrier molecules, retardants, and other excipients. In particular, peptides and (poly)peptides as specified may be comprised in fusion polypeptides comprising further peptides, which may serve e.g. as a tag for purification and/or detection, as a linker, or to extend the in vivo half-life of a compound.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually considered to be “peptides”. The peptides referred to herein are disclosed as SEQ ID NOs:1 to 6, as well as in Table 1 herein below. As the skilled person understands from the disclosure herein, the immunization peptides described herein are preferably used with the HLA supertypes indicated herein in Table 1.

The terms “human papillomavirus 16-related virus” and “HPV16-related virus” relate to any virus, preferably infecting humans, encoding at least one amino acid sequence of SEQ ID NOs: 1 to 6. Preferably, the HPV16-related virus is a papillomavirus (PV), more preferably a human papillomavirus (HPV), still more preferably is selected from the list consisting of HPV16, HPV31, HPV33, HPV35, HPV52, HPV58, and HPV67, most preferably is HPV16.

The term “immunization peptide”, as used herein, relates to a discrete peptide consisting of an amino acid sequence selected from SEQ ID NOs:1 to 6. Preferably, the immunization peptide has been verified to activate human anti-immunization peptide T cells when presented as a human lymphocyte antigen (HLA)-complex. Also preferably, a HPV16-derived peptide consisting of the same amino acid sequence as the at least one immunization peptide has been verified to be presented by human HPV16-positive tumor cells. Thus, preferably, the immunization peptide is a peptide verified to induce activation of immune cells and to be presented on cancer cells infected with HPV16.

The term “vaccine” is understood by the skilled person to include each and every agent eliciting an immune response in a subject. Preferably, said immune response is a cellular immune response, more preferably a T cell response, even more preferably a CD8+ T cell response, most preferably a cytotoxic T cell response. As the skilled person understands, the immunization peptides as referred to herein are derived from HPV16 E6 or E7, which are oncogenic HPV polypeptides; in accordance, the vaccine preferably is a therapeutic vaccine. Preferably, the vaccine comprises a nanoemulsion comprising said immunization peptides or comprises silica nanoparticles, preferably with a size of at most 150 nm. Preferably, in the vaccine, the immunization peptide(s) are linked to said nanoparticles, preferably by an adsorptive bond.

Preferably, the vaccine is a pharmaceutical composition, optionally comprising a carrier; preferably, the carrier is a pharmaceutically acceptable carrier. In addition, vaccine may also include other additional compounds, such as adjuvants, stabilizers and/or other compounds deemed appropriate by the skilled person, e.g. for galenic purposes. As referred to herein, the immunization peptides as specified herein above, are the “active compound” of the vaccine, although further active compounds may be present. Vaccine compositions are preferably prepared in a manner well known in the pharmaceutical art. Formulations are to be adapted to the mode of administration, i.e. in the forms of tablets, capsules, suppositories, solutions, suspensions or the like. Dosage recommendations shall be indicated in the prescriber's or user's instructions in order to anticipate dose adjustments depending on the considered recipient.

Preferably, the vaccine further comprises an adjuvant. More preferably, the immunization peptides and the adjuvant are comprised in a common mixture at administration. Thus, preferably, the immunization peptide and the adjuvant are mixed before administration. Preferably, the adjuvant comprises a T-helper-1 response eliciting substance, such as e.g. a TLR-3 agonist (e.g. polyinosinic:polycytidylic acid, (poly(1:C)) or any derivatives thereof wherein said derivative preferably is a polyl:polyC12U or poly-ICLC (Hiltonol™). Thus, the adjuvant preferably is an anionic compound as specified elsewhere herein. However, also other adjuvants known to the skilled person may be used.

The term “eliciting an immune response” is understood by the skilled person and includes an increase of the extent of at least one feature of an immune response of a human subject to a non-self antigen. Thus, the term includes initiating a de novo response as well as enhancing an existing immune response. Preferably, eliciting an immune response comprises administering a vaccine providing the immunization peptides as specified herein above to a subject. Thus, the immunization peptide preferably is presented via a major histocompatibility complex (MHC) class I molecule, preferably on a nucleated cell, more preferably on an antigen presenting cell and/or on a nucleated cell infected with a HPV16-related virus, e.g. cells of an HPV16-related virus-positive inappropriate cellular proliferation, in particular a HPV16-related lesion or HPV16-related cancer as specified elsewhere herein. Thus, eliciting an immune response preferably is eliciting an immune response to a HPV16-related virus-positive inappropriate cellular proliferation. As usual in the art, MHCs may also be referred to as human lymphocyte antigens (HLAs), of which HLA-A, -B, and -C are MHC class I types. Preferably, eliciting an immune response comprises presentation of the immunization peptide on at least one HLA type belonging to one of HLA supertypes A1, A2, A3/A11, A24, B7, and B15.

Eliciting an immune response as referred to herein may comprise administration of a vaccine providing the immunization peptides to a subject as specified herein below, which proceeding may also be referred to as vaccination, in particular vaccination against HPV16-related virus infection. The term “vaccination” and in particular the term “vaccination against HPV16-related virus infection” as used herein, preferably, relates to administering a vaccine as specified herein to elicit an immune response against HPV16-related virus genotypes, in particular directed against cells infected therewith. Thus, vaccination preferably stimulates the immune system and establishes or improves immunity to infection with various HPV16-related virus genotypes. Preferably, vaccination according to the present invention allows for establishing or improving immunity to infection with HPV16-related virus genotypes. It is to be understood that the vaccine according to the present invention may comprise further components, in particular as specified elsewhere herein. The skilled person will understand that vaccination may not elicit a significant immune response in all subjects vaccinated. Also, it is to be understood that vaccination may not be effective to treat or prevent infection in all subjects vaccinated. However, the term requires that a, preferably statistically significant, portion of subjects of a cohort or population are effectively vaccinated, wherein effective vaccination, preferably, is prevention or reduction of the number of HPV16-related virus-induced lesions or reduction of HPV16-related cancer mass and/or volume. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 60%, at least 70%, at least 80%, or at least 90% of the subjects of a given cohort or population. The aforesaid vaccination may be prophylactic, i.e. vaccination in a narrow sense, and provide preventing or reducing infection and/or ameliorating consequences of infection; or may be therapeutic, i.e. provide reduction or clearance of existing infection. Thus, in the context of HPV16-related virus-positive inappropriate cellular proliferations, therapeutic vaccination may in particular cause a reduction in number or complete clearance of cells infected with and/or transformed by said HPV16-related virus. In accordance, eliciting an immune response to a HPV16-related virus is eliciting an immune response to a HPV16-related virus infection, in particular to HPV16 E6 and/or E7 proteins.

The term “vaccine providing immunization peptides”, as referred to herein, relates to any composition of matter causing the six immunization peptides to become present, in particular after its application to a cell, preferably to a subject. Preferably, providing is causing immunization peptides to become present by processes occurring naturally in a host cell, in particular proteasome proteolysis and/or MHC class I presentation pathways. Thus, the vaccine providing immunization peptides may be any mixtures of peptides each comprising an immunization peptide; however, as for the immunization peptide, also the vaccine providing an immunization peptide comprises at most 100, preferably at most 50, more preferably at most 25, most preferably at most 12, contiguous amino acids of a sequence of an HPV16 E6 or E7 polypeptide. Preferably, at least one immunization peptide is linked in said vaccine to at least one of a spacer, a linker, an anionic compound, and a nanoparticle, all of which are in principle known to the skilled person. From such a vaccine providing immunization peptides, the immunization peptide may be liberated, e.g. by proteolysis (e.g. by a proteasome), by hydrolysis, e.g. of an amido or ester bond to a carrier molecule or by desorption. Corresponding compositions and methods are known in the art, in particular from EP 20 174 156, EP 20 196 027, and EP 20 196 024. Also preferably, the vaccine providing an immunization peptide may be a composition of matter comprising at least one immunization peptide as such. Preferably, the immunization peptide is presented by a host cell via major histocompatibility complex (MHC) class I.

The term “linker”, as used herein, relates to any and all chemical structures connecting to elements, preferably of a peptide or a polypeptide. Preferably the linker comprises at least one functional group being a carboxyl (—COOH) or carboxylate (—COO—) group and/or at least one functional group being a guanidino-group (—NHC(═NH)NH₂ or —NHC(═NH₂⁺)NH₂) or amino-group (—NH₂ or —NH₃⁺). More preferably, the linker comprises both a carboxyl (—COOH) or carboxylate (—COO—) group and at least one group selected from guanidino or amino. Even more preferably, the linker comprises a carboxyl or carboxylate group and at least one guanidino-group.

The “anionic compound” preferably is a pharmaceutically acceptable compound comprising polyinosinic:polycytidylic acid, (poly(1:C)) or any derivatives thereof wherein said derivative preferably is a polyl:polyC12U and poly-ICLC.

The term “spacer” is known to the skilled person. Preferably, the spacer is a self-immolative spacer, i.e. spontaneously disassembling. Preferably, the spacer comprises a structural unit comprising 4-aminobenzyl alcohol, 2-aminobenzyl alcohol, 4-hydroxybenzyl alcohol, or 2-hydroxybenzyl alcohol, as reported in e.g. EP 0 648 503, and WO 2015/162291 A1.

The vaccine providing immunization peptides provides a multitude of immunization peptides, i.e. at least the six immunization peptides specified herein above which bind to non-identical HLA supertypes selected from the list consisting of HLA supertypes A1, A2, A3/A11, A24, B7, and B15, wherein the term “A3/A11” relates to a common supertype of HLA-A3 and HLA-A11 types. The term “HLA supertypes” is known to the skilled person, e.g. from Sidney et al. (2008), BMC Immunology 9 Art. No. 1, doi.org/10.1186/1471-2172-9-1, where supertype B15, however, is referred to as B62.

In view of the above, the vaccine preferably is a universal vaccine providing immunization peptides such that at least 90%, preferably at least 95%, even more preferably at least 98%, most preferably at least 99%, of subjects of the world's population express at least one HLA supertype capable of presenting at least one of said immunization peptides. Thus, the HLA universal vaccine preferably comprises a mixture of immunization peptides presented by HLA molecules of the most frequent HLA supertypes; frequencies of HLA supertypes are known in the art and are summarized herein below in Table 3.

The term “subject”, as used herein, relates to a mammal, preferably a human. Preferably the subject comprises at least one HLA type as specified herein below in Table 1. Preferably, the subject is infected with at least one HPV16-related virus. Preferably, the subject is afflicted with at least one HPV16-related virus positive inappropriate cellular proliferation. Also preferably the types of HLA molecules expressed by said human subject are unknown. Also preferably, the subject is known or suspected to express at least one of HLA molecule from at least one supertype selected from the list consisting of HLA-A01, HLA-A02, HLA-A3/A11, HLA-A24, HLA-B07, and HLA-B15. As will be understood by the skilled person from the disclosure herein, more than 90% of subjects in an average caucasoid, oriental, mixed, amerindian, african, or asian population, express at least one of the aforesaid HLA supertypes, as shown in Table 3. Thus, a subject preferably is suspected to express at least one HLA from one of said HLA supertypes unless it is known, e.g. from HLA typing, not to do so.

Advantageously, it was found in the work underlying the present invention that HLA presentation of HPV16-derived peptides by HPV16-positive cells can be evaluated by mass spectrometry as disclosed in the Examples, providing experimental proof that peptides are in fact presented to the immune system on the intended target cells. Also, immunogenicity assays as shown in the Examples, performed on peripheral blood lymphocytes (PBMCs) of various donors can be used to provide proof that respective peptides are indeed active in vivo in inducing immune responses. Thus, in order to provide actionable target structures for vaccination against HPV16-mediated malignancies (therapeutic vaccines, adoptive transfer of T cells recognizing an immunization presented via a HLA on a target cell), it was found relevant to validate T cell epitope/HLA complexes as actionable targets to ensure efficacy of the above mentioned therapies. Preferably, it may not be sufficient to use HLA binding prediction algorithms to define actionable targets, as these are not precise enough and miss many binding peptides, cf Table 2. Thus, not only HLA binding of each stimulation peptide referred to herein was experimentally validated, but, furthermore, stimulation peptides were assessed with PBMCs from healthy human donors for T cell memory responses. The thus defined immunogenic peptides must have been naturally processed and presented in a prior HPV16 infection. Also, it is preferred to validate the HLA presentation of putative stimulation peptides directly on the tumor (or other HPV-positive) cells. This can be achieved by a mass-spectrometry-based technology, immunopeptidomics. Thus, in particular the aforesaid two methods in combination can identify good candidate peptides for eliciting a cellular immune response against HPV16-related virus-infected cells and inappropriate cellular proliferations caused by them.

The present invention further relates to a vaccine of the present invention for use in medicine, and/or for use in eliciting an immune response in a human subject against an HPV16-related infection, wherein said eliciting an immune response preferably is treating and/or preventing HPV16-related inappropriate cellular proliferation.

The term “inappropriate cellular proliferation” relates to an abnormal proliferation of body cells in a subject; as a consequence, an imbalance of cellular composition of a body tissue, of a body fluid and/or tumor formation may ensue. Inappropriate cellular proliferation may be induced by an infectious agent, preferably a virus, more preferably an HPV16-related virus. Preferably, inappropriate cellular proliferation is benign, i.e. preferably, does not threaten health or life of a subject. Preferred benign inappropriate cellular proliferations are warts, exophytic growing papillomas, condylomata, inverted papillomas, and pre-neoplastic HPV-induced lesions. Also preferably, the inappropriate cellular proliferation is pre-malignant or malignant, i.e. does at least potentially threaten health or life of a subject; thus, preferably, the inappropriate cellular proliferation is a pre-malignant or malignant inappropriate proliferation of a mucosa or a skin, in particular a mucosa, e.g. of the oropharynx, anogenital regions, and/or reproductive organs, e.g. cervical intraepithelial neoplasia (CIN) or cancer, in particular cervical cancer and/or head and neck cancer. The term “cancer”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is a relapse. Thus, preferably, the cancer is a solid cancer, a metastasis, or a relapse thereof. Thus, eliciting an immune response preferably is cancer prevention and/or cancer treatment.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., as described herein below. Preferably, treating cancer is reducing tumor burden in a subject. As will be understood by the skilled person, effectiveness of treatment of e.g. cancer is dependent on a variety of factors including, e.g. cancer stage and cancer type. Preferably, treating causes inappropriately proliferating cells, preferably neoplastic cells, more preferably cancer cells, to be recognized by T-cells of the subject. Thus, preferably, treating has the effect of killing tumor cells, causing a tumor to stop growing, in particular causing tumor cells to stop proliferating, more preferably causing regression of a tumor, more preferably causing a tumor to resolve. As used herein, the above relates to treating other HPV16 related virus-positive inappropriate cellular proliferations mutatis mutandis.

The term “preventing” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification. Thus, preferably, in case of disease caused by an infectious agent, preventing may be vaccination. Thus, preferably, the term preventing relates to administering the compounds as specified herein to elicit an immune response against at least one infectious agent.

The present invention also relates to a method of inducing an immune response to a HPV16-related virus in a subject, comprising contacting said subject with a vaccine of the present invention.

The present invention further relates to a use of a vaccine of the present invention in the manufacture of a medicament, preferably a vaccine. The present invention further relates to a use of a vaccine of the present invention in the manufacture of a medicament, preferably a vaccine.

The present invention moreover relates to a kit comprising the vaccine of the present invention comprised in a housing.

The term “kit”, as used herein, refers to a collection of the aforementioned compounds, means or reagents. The components of the kit may be comprised by separate vials (i.e. as a kit of separate parts) or provided in a single vial, e.g. as a composition as specified herein above. The housing of the kit preferably allows translocation of the compounds of the kit, in particular common translocation; thus, the housing may in particular be a transportable container comprising all specified components. Moreover, it is to be understood that the kit of the present invention may be used for practicing at least one of the methods referred to herein above. It is envisaged that all components may be provided in a ready-to-use manner for practicing a method referred to above. Further, the kit preferably contains instructions for carrying out said methods, e.g. dosage instructions. The instructions can be provided by a user manual in paper- or electronic form. The kit preferably comprises further components, preferably at least one further therapeutic agent as specified herein above, an excipient, and/or a means of administration, in particular a syringe and/or a needle, and/or an IV infusion equipment.

Also, the present invention relates to a device comprising the vaccine of the present invention.

The term “device”, as used herein relates to a system of means comprising at least the means operatively linked to each other as to allow administration of the vaccine and/or the vector. Preferred means for administering polynucleotides, compositions, or viruses are well known in the art. How to link the means in an operating manner will depend on the type of means included into the device and on the kind of administration envisaged. Preferably, the means are comprised by a single device in such a case. Said device may accordingly include a delivery unit for the administration of the compound or composition and a storage unit for storing said compound or composition until administration. However, it is also contemplated that the means of the current invention may appear as separate devices in such an embodiment and are, preferably, packaged together as a kit. The person skilled in the art will realize how to link the means without further ado. Preferred devices are those which can be applied without the particular knowledge of a specialized technician. Preferably, the device is a syringe, more preferably with a needle, comprising the compound or composition of the invention. Also preferably, the device is an intravenous infusion (IV) equipment comprising the compound or composition. Also preferably, the device is an endoscopic device comprising the compound or medicament for flushing a site of administration, or further comprising a needle for topical application of the compound or composition, e.g. to a tumor. Also preferably, the device is an inhaler comprising the compound of the present invention, wherein, more preferably, said compound is formulated for administration as an aerosol. Further preferably, the device is a cervical cap, a swab, a tampon, a vaginal ring, a vaginal strip, a vaginal capsule, a bioadhesive film, a sponge or a brush; in such case, the vaccine and/or vector may in particular be formulated as a cream, emulsion, nanoemulsion, or the like.

The present invention also relates to a method for stimulating T cells specifically binding to human host cells infected with a HPV16-related virus (anti-HPV T cells), said method comprising
(A) contacting an agent expressing MHC class I molecules with a vaccine according to the present invention, thereby causing presentation of at least one of said immunization peptides on said MHC class I molecules, and
(B) contacting the agent presenting MHC class I molecules of step (i) with T cells, thereby stimulating anti-HPV T cells.

In view of the above, the following embodiments are particularly envisaged:

Embodiment 1: A vaccine against a human papillomavirus 16 (HPV16)-related virus providing at least six discrete immunization peptides consisting of the amino acid sequences of SEQ ID NOs:1 to 6, wherein said vaccine comprises a mixture of discrete peptides each comprising exactly one of said amino acid sequences.

Embodiment 2: The vaccine of embodiment 1, wherein at least one, preferably all, of said immunization peptides are linked to at least one of a spacer, a linker, and a nanoparticle.

Embodiment 3: The vaccine of embodiment 1 or 2, wherein said nanoparticle is a silica nanoparticle with a size of at most 150 nm.

Embodiment 4: The vaccine of any one of embodiments 1 to 3, wherein said at least one immunization peptide is linked to said nanoparticle, preferably via said spacer and/or linker, by an adsorptive bond.

Embodiment 5: The vaccine of embodiment 4, wherein said linker comprises at least one functional group being a carboxyl, carboxylate, guanidino, or amino-group.

Embodiment 6: The vaccine of embodiment 4 or 5, wherein said spacer is a self-immolative spacer.

Embodiment 7: The vaccine of any one of embodiments 1 to 5, further comprising an anionic compound, preferably polyinosinic:polycytidylic acid, (poly(1:C)), or any derivatives thereof.

Embodiment 8: The vaccine of any one of embodiments 1 to 7, further comprising an adjuvant, a carrier, and/or a stabilizer.

Embodiment 9: The vaccine of any one of embodiments 1 to 8, wherein said HPV16-related virus is selected from the list consisting of HPV16, HPV31, HPV33, HPV35, HPV52, HPV58, and HPV67.

Embodiment 10: A vaccine according to any one of embodiments 1 to 9 for use in medicine.

Embodiment 11: A vaccine according to any one of embodiments 1 to 9 for use in eliciting an immune response in a human subject against HPV16-related virus infection.

Embodiment 12: The vaccine for use of embodiment 11, wherein said eliciting an immune response is treating and/or preventing HPV16-related virus infection and/or HPV16-related inappropriate cellular proliferation.

Embodiment 13: The vaccine for use of embodiment 11 or 12, wherein said subject is infected with at least one HPV16-related virus.

Embodiment 14: The vaccine for use of any one of embodiments 11 to 13, wherein the HLA molecules produced by said subject are unknown.

Embodiment 15: The vaccine for use of any one of embodiments 11 to 14, wherein said subject is known or suspected to express at least one HLA molecule of at least one of the HLA supertypes selected from the list consisting of HLA-A01, HLA-A02, HLA-A3/A11, HLA-A24, HLA-B07, and HLA-B15.

Embodiment 16: A method of inducing an immune response to a HPV16-related virus in a subject, comprising contacting said subject with a vaccine according to any one of embodiments 1 to 9.

Embodiment 17: Use of a vaccine according to any one of embodiments 1 to 9 in the manufacture of a medicament, preferably a vaccine.

Embodiment 18: A kit comprising the vaccine according to any one of embodiments 1 to 9 comprised in a housing.

Embodiment 19: The kit of embodiment 18, further comprising a means of administration.

Embodiment 20: A device comprising the vaccine according to any one of embodiments 1 to 9.

Embodiment 21: A method for stimulating T cells specifically binding to human host cells infected with a HPV16-related virus (anti-HPV T cells), said method comprising
(A) contacting an agent expressing MHC class I molecules with a vaccine according to any one of embodiments 1 to 9, thereby causing presentation of at least one of said immunization peptides on said MHC class I molecules, and
(B) contacting the agent presenting MHC class I molecules of step (i) with T cells, thereby stimulating anti-HPV T cells.

Embodiment 22: A method for treating HPV16-related virus infection or treating and/or preventing HPV16-related virus positive inappropriate cellular proliferation in a human subject, comprising
(I) administering a vaccine according to the present invention to said human subject; and
(II) thereby treating said HPV16-related virus infection or treating and/or preventing said HPV16-related virus positive inappropriate cellular proliferation.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

### EXAMPLE 1: METHODS FOR T CELL REACTIVITY ASSESSMENT

### Interferon-γ ELISpot Assay

Prior to the ELISpot assay, ELISpot plates (Millipore Multiscreen-HA membrane sterile plate) were coated with 100 μl of 2 μg/mL anti-human IFN-γ (1-D1K) antibody in sterile PBS and incubated at 4° C. overnight or up to 3 days.

Before setting up the ELISpot assay, the coating antibody was discarded, wells were washed three times with 200 μL sterile PBS and blocked with 200 μL ELISpot medium at standard culture conditions for 1-1.5 h. After blocking, ELISpot medium was discarded and wells were filled with 100 μL antigen solution.

Each (short-term) T cell line (see below) was stimulated in a total of 8 wells: four wells with respective antigen (10 μg/mL single peptides, 0.1% DMSO or CEF peptide pool (1 μg/mL of each peptide)), two wells with concanavalin A (2 μg/ml) as unspecific mitogen-stimulated positive control, and two wells with 0.1% (v/v) DMSO as background control. Short-term antigen-specific T cell lines (described in 1.4) were added in a concentration of 1-2×10⁵ cells in 100 μL per well (total volume of 200 μL/well). The cell numbers of three representative wells (CEF-, DMSO-, peptide-stimulated) were assessed, to enable the calculation of spot forming units (SFU) per 1×10⁶ cells later on. The ELISpot plates were incubated at standard culture conditions for 20-24 h. Moving the plates or the incubator was strictly avoided during incubation to prevent blurred spot formation.

The remaining cell suspension was discarded and ELISpot plates were washed twice with PBS and twice with ELISpot washing buffer. All washes were performed with 200 μL/well. Then, 100 μL/well of 1 ng/mL sterile anti-human IFNγ biotin (7 B6-1) antibody in sterile PBS were added. After two hours of incubation in the dark at room temperature, the antibody was discarded and the plates were washed four times with ELISpot washing buffer. Sterile streptavidin-alkaline phosphatase was diluted by a factor of 1:2000 in sterile PBS and 100 μL/well of this dilution were distributed.

After 1.5 hours of incubation in the dark at room temperature, the antibody was discarded and the plates were washed four times with ELISpot washing buffer. Then, the BCIP/NBT-Plus substrate was filtered through a 0.22 μm filter and 100 μL/well were distributed. The plates were covered with aluminum foil and left at room temperature for 18-20 min until spots developed in the positive control wells. The reaction was stopped by washing the ELISpot plates with tap water and the plates were allowed to air dry for a minimum of 1.5 h.

### Analysis of ELISpot Plates

In order to obtain objective results about the number of SFU per well, a CTL automated ELISpot plate reader was used to count the ELISpot plates. The SmartCount™ counting mode of the ImmunoSpot 5.1.36 Professional DC software was used for the analysis. The settings were adjusted for each donor individually to account for donor specific variance. In general, the background balance was set to values between 0 and 10 and the spot separation was set to values between 1 and 4. The automatically determined minimum spot size was adjusted to exclude small background spots based on negative control wells. Similarly, maximum spot size was automatically determined based on positive control wells. A quality control for each well was done manually to detect and correct errors of the counting algorithm caused by uneven development or fibers. Based on the counting results, the mean SFU per 1×10⁶ cells and the stimulation index (SI), defined as the fold change of the mean SFU of peptide-stimulated relative to DMSO background wells, were calculated for each peptide-specific cell line. Responses with SI >2 and SFU per 1×10⁶ cells >100 as well as responses with SI >4 and SFU >50 were considered positive.

### T Cell Phenotyping and Intracellular Cytokine Staining

To characterize and quantify the responsive cells upon peptide stimulation, the peptide-specific T cell cultures with positive ELISpot results were further analyzed with T cell phenotyping and intracellular cytokine staining (ICS) assessed by flow cytometry.

The cells of the respective continued short-term T cell culture as well as the DMSO-stimulated and an unresponsive T cell line were harvested into 2 mL reaction tubes and centrifuged at 400×g for 10 min. The supernatant was discarded and the pellet of the unresponsive T cell line was resuspended in 100 μL ELISpot medium and used as unstimulated and unstained control. The pellets of the responsive and the DMSO-stimulated T cell lines were resuspended in 200 μL ELISpot medium and split equally into two wells of a V-bottom 96-well plate. One well of the responsive T cell lines was re-stimulated with 10 μg/mL of its specific peptide while the other well was mock stimulated with 0.1% (v/v) DMSO. As positive control, one well of the DMSO-stimulated T cell line was activated by treatment with 20 ng/ml Phorbol 12-myristate 13-acetate and 1 μM Ionomycin. Then, 1:10 diluted GolgiStop (containing monensin) and 1:15 diluted GolgiPlug (containing brefeldin A) were added to all wells to inhibit intracellular transport processes. Monensin inhibits the trans-Golgi transport, while brefeldin A inhibits transport between the ER and the Golgi (Chardin and McCormick, 1999; Mollenhauer et al., 1990). The stimulated cells were incubated for 5 h at standard culture conditions.

After incubation, the cells were resuspended, centrifuged at 400×g for 5 min and the supernatant was discarded. Then, the cells were resuspended in 50 μL cold staining buffer (unstained) or 50 μL cold staining buffer containing the phenotyping antibodies against CD3, CD4, CD8 and the LIVE/DEAD™ Fixable Near-IR Dead Cell Stain (unstimulated and stimulated) and incubated at 4° C. for 30 min. After the surface staining, the cells were washed twice with 200 μL staining buffer and centrifugation at 1400 rpm, followed by fixation with 1% paraformaldehyde (PFA) solution for 15 min at 4° C. All following centrifugation steps were carried out at 1400 rpm for 5 min. After fixation, the cells were again washed twice with 200 μL cold staining buffer and were then resuspended in 100 μL 1× perm/wash buffer diluted in PBS (provided is 10×) for 15 min at 4° C. for cell membrane permeabilization. Subsequently, the plate was centrifuged and the supernatant was discarded. The cells were resuspended in 50 μL perm/wash buffer diluted in PBS (unstained) or 50 μL perm/wash buffer containing the intracellular cytokine antibodies against IFNγ, TNFα and granzyme B (unstimulated and stimulated) for 30 min at 4° C. After intracellular cytokine staining, the cells were washed twice with 200 μL 1× perm/wash buffer. Then 100 μL fix/perm solution (provided in the BD Cytofix/Cytoperm kit) were added per well. After 20 min of incubation at 4° C., the cells of each well were washed twice with 200 μL 1× perm/wash buffer before being resuspended in 100 μL staining buffer and stored at 4° C. overnight.

OneComp eBeads™ (eBeads) and ArC™ Amine Reactive Compensation beads (ArC beads) were stained as compensation controls. For each antibody, 50 μL of the eBeads suspension were stained with the same dilution as used for staining the cells. The ArC beads were stained with 1 μL LIVE/DEAD™ Fixable Near-IR Dead Cell Stain stock solution in 50 μL staining buffer. The beads were incubated at 4° C. for 30 min and were then washed two times with 1 mL staining buffer and centrifugation at 400×g. Finally, the stained beads were resuspended in 400 μL staining buffer, one drop of ArC™ negative beads was added to the labelled ArC beads, and all beads were stored at 4° C. overnight.

On the next day, the samples and the single stained compensation controls were acquired at a BD FACS Canto™ II analyzer using the BD FACS Diva Software Version 6. The obtained data were analyzed with the FlowJo software Version 10 by applying the same gating strategy to all samples.

### Short-Term T Cell Lines

Short-term antigen-specific T cell lines were generated from PBMCs in order to expand antigen-specific memory T cells induced by previous natural HPV16 infections. To do so, T cells were stimulated with HLA-matched HPV16 E6- or E7-derived HLA-ligands. To set up short-term T cell lines, human PBMCs were thawed and subsequently resuspended in T cell medium supplemented with recombinant human interleukin-7 (rhIL-7) (10 ng/mL) and recombinant human interleukin-15 (rhIL-15) (20 ng/mL). The cells were counted and 1-2×10⁶ cells per antigen stimulation were seeded in wells of 24-well plates in a total volume of 2 mL per T cell line. To obtain antigen-specific T cell lines, 10 g/mL of HLA-binding HPV16 E6/E7-derived peptides were added to each well. As peptide-specific positive control, one cell line was stimulated with a peptide pool consisting of 23 well described HLA class I-restricted epitopes of the widespread viruses human cytomegalovirus (CMV), Epstein-Barr-Virus (EBV) and Influenza A (CEF peptide pool) (Currier et al., 2002) at a concentration of 1 μg/mL for each peptide. As single peptide controls, two cell lines were stimulated with 10 g/mL HLA-matched EBV- and CMV-derived peptide. As peptide-specific negative control, a cell line was stimulated with 10 μg/mL HLA-matched human immunodeficiency virus (HIV)-derived peptide (10 μg/mL), while treatment with 0.1% (v/v) dimethyl sulfoxide (DMSO, peptide solvent) served as unspecific negative control. The stimulated PBMCs were cultivated at standard culture conditions.

On day 3, the cells were fed with recombinant human interleukin-2 (rhIL-2) (20 U/mL) and rhIL-15 (20 ng/mL) to promote proliferation of T cells. Seven days after culture setup, a half-medium change was performed. For this, the cells were pelleted on the well bottom by centrifugation at 300×g for 5 min and 1 mL of the supernatant was carefully removed from each well. Then, 1 ml fresh T cell medium supplemented with rhIL-2 (20 U/mL final) and rhIL-15 (20 ng/mL final) was added and cells were resuspended.

After 12 days of culturing, half of the cells of each culture was used to set up ELISpot assays as described in 1.1. The remaining cells were fed with 1 mL ELISpot medium and cultivated for two more days until the cells were used for T cell phenotyping and intracellular cytokine staining (ICS) as described in 1.2.

### Cytotoxicity Assay

The flow cytometry based VITAL FR cytotoxicity assay was used as published by Stanke et al. (Stanke et al., 2010) to assess whether peptide-specific T cells are able to specifically kill HPV-transformed target cells. Briefly, two HLA-A2⁺ cell lines, a HPV16-negative (HPV16⁻) control cell line (C33A) and a HPV16-positive (HPV16⁺) target cell line (CaSki) were fluorescently labeled with either FarRed or CFSE and were co-incubated with peptide-specific CD8⁺ T cells as effector cells. After 48 h co-incubation, the fluorescently labeled cells were analyzed by flow cytometry to determine the surviving cells and calculate the specific cytolytic function of the effector cells by the ratio of target and control cells.

CaSki and C33A cells were fluorescently labeled with CFSE and FarRed, respectively. Cells of both cell lines were harvested and resuspended in plain RPMI medium at a concentration of 1×10⁶ cells/mL. For cell labeling, 5 μM CSFE were added to CaSki cells and 0.25 μM FarRed were added to C33A cells. The cells were incubated at 37° C. and the reaction was stopped after 10 min by addition of 50 mL RPMI with 10% FBS. The cells were centrifuged at 300×g for 5 min, the supernatant was discarded and the cells were resuspended in their cell culture medium. The labeled cells were seeded in T25 cell culture flasks with 1-2×10⁶ cells/flask.

On the next day, the labeled CaSki and C33A as well as the epitope-specific semi-long-term T cell lines (described in 1.6) were harvested. CD8⁺ T cells were isolated from the epitope-specific semi-long-term T cell lines using the MACS CD8⁺ T cell isolation kit (Miltenyi Biotec) according to the manufacturer's protocol for LS columns. The CD8⁺ T cells were resuspended in T cell medium and added to wells of a F-bottom 96-well plate in serial dilutions to achieve triplicates of 6×10⁴, 3×10⁴, 1.5×10⁴, 0.75×10⁴ and 0.375×10⁴ cells per well. The CaSki and C33A cells were mixed 1:1 in T cell medium and 3×10³ cells were added to each well containing T cells. Thereby effector:target (E:T) ratios of 1:20, 1:10, 1:5, 1:2.5 and 1:1.25 were achieved. Additionally, triplicates of mixed target cells without T cells were seeded, which is referred to as E:T 0. After 48 h incubation at 37° C., 5% CO₂, the cells were harvested and analyzed at a BD FACS Canto™ II analyzer using the BD FACS Diva Software Version 6. The obtained data were analyzed with the FlowJo software Version 10 by applying the same gating strategy to all samples. Subsequently, the equations (1) and (2) given below were applied in Excel (Microsoft Office 2016) to calculate specific killing.

$$ratio of target cells ( \% ) = \frac{frequency of CaSki}{frequency of C 33 A} \qquad ( 1 )$$

$$( 2 )$$

$$specific killing ( \% ) = 100 - ( \frac{ratio of target cells in presence of T cells}{ratio of target cells without T cells} * 100 )$$

### Semi Long-Term T Cell Lines

Semi long-term T cell lines were set up to obtain high numbers of T cells, which are required for the analysis of epitope specific cytotoxic T cell responses in VITAL FR cytotoxicity assays. Autologous DCs were generated from PBMCs as described in 1.7 and loaded with the desired HPV16 peptide. PBMCs of the same donor were thawed and resuspended in T cell medium supplemented with rhIL-7 (10 ng/mL) and rhIL-15 (20 ng/mL). 1×10⁷ PBMCs/well were seeded to a 24-well plate in a total volume of 1 mL/well. Peptide-loaded DCs were added in a ratio of 50:1 (PBMCs:DCs) in 1 mL rhIL-7 (10 ng/mL) and rhIL-15 (20 ng/mL) supplemented T cell medium to achieve a final volume of 2 mL. The cell cultures were fed with rhIL-2 (final concentration: 40 U/mL) every other day. If the medium turned yellow the feeding was combined with a half medium change with 1 mL fresh TL-2 supplemented T cell medium. After eight days, the peptide stimulation with peptide-loaded DCs was repeated and cultivation was continued. On day 15, the T cell lines were harvested and used for CD8⁺ T cell isolation and cytotoxicity assays.

### Generation of Autologous DCs

To generate autologous DCs, frozen human PBMCs were thawed. After thawing, cells were counted and resuspended in DC medium to reach a density of 5×10⁶ cells/mL. From this suspension, 1×10⁷ cells in 2 mL DC medium were seeded per well in a 6-well plate.

After 3 hours incubation at 37° C., 5% CO₂ the supernatant was removed and transferred to a 50 mL tube. The wells were gently washed with 1 mL warm DC medium to remove non-adherent cells and the washing medium was collected together with the supernatant. The remaining adherent cells were fed with 2 mL fresh DC medium supplemented with 100 ng/mL granulocyte-macrophage colony-stimulating factor (GM-CSF) and 50 ng/mL interleukin-4 (IL-4) for further cultivation. The collected non-adherent cells containing T and B cells were counted and frozen for future use.

After three days of culturing, the cells were fed with 0.5 mL fresh DC medium supplemented with 100 ng/mL GM-CSF and 50 ng/mL TL-4. Six days after the cultures were started, the DCs were maturated by adding 1000 U/ml tumor necrosis factor α (TNFα), 10 ng/ml interleukin-1β (IL-1β), 10 ng/ml interleukin-6 (IL-6), 1 μM Prostaglandin E2 (PGE2) and 1 μg/ml lipopolysaccharide (LPS) to each well. The cells were incubated with this maturation cocktail for 48 hours and harvested by gentle scraping with a cell scraper.

### EXAMPLE 2: IMMUNOPRECIPITATION (IP) AND LC/MS ANALYSIS OF PEPTIDES PRESENTED BY Cultured Cells

### Immunoprecipitation of HLA-Presented Peptides

HLA immunoprecipitation (IP) was performed according to previously published protocols (Bassani-Sternberg et al., 2016; Chong et al., 2018). Briefly, HPV16+ cells were lysed with a lysis buffer containing 1% N-octyl-β-D glucopyranoside, 0.25% Na-Deoxycholate, protease inhibitor cocktail (Sigma-Aldrich, Mannheim, Germany)/PMSF (Carl Roth, Karlsruhe, Germany) in PBS. After centrifugation at 40,000×g, 4° C., for 30 min. HLA-peptide complexes were immuno-precipitated by incubation with mouse anti-human HLA-A,B,C monoclonal antibody (clone W6/32, Biolegend, San Diego, CA, USA) or in some cases HLA-type-specific antibody (HLA-A2: clone BB7.2, HLA-A3: clone GAP A3, HLA-A11/A24: clone Hb 164, HLA-B7: clone BB7.1) crosslinked to protein G Sepharose beads (Cytiva, Marlborough, MA, USA) or Protein A—Sepharose™ 4B (Invitrogen Corporation, Camarillo, CA, USA) for 4 h at 4° C. under constant mixing on a rotating wheel. Supernatant was discarded after centrifugation at 3200×g, for 3 min at RT. Pelleted HLA-peptide complexes bound to antibody-beads were washed 3 times in each of the following steps: first with ice-cold 20 mM Tris-HCl pH 8 containing 150 mM NaCl followed by the same buffer but with additional 400 NaCl and finally with 20 mM Tris-HCl alone. Peptides were eluted from antibody-beads bound to HLA by 0.3% TFA. Resulting peptides were desalted by reverse-phase purification using a SepPak 96-well plate (Waters, Milford, MA, USA). Peptides eluted in 28% ACN in 0.1% TFA were dried by vacuum centrifugation (Concentrator plus, Eppendorf, Hamburg, Germany).

### Direct Infusion and LC-MS

The set of predicted target peptides were acquired as stable isotope labelled (SIL) peptides from commercial suppliers (crude synthesis product; isotope purity >99 atom % ¹³C and ¹⁵N). First, analysis by direct infusion was performed in order to optimize normalized collision energy (NCE) for each single peptide in a mixture. NCE values were ranging from 4% to 42% and each value, in 2% steps, was measured 4 times for four charge states (1+ to 4+) of each peptide. Orbitrap Exploris 480 (Thermo Fisher Scientific) in targeted MS2 scan (PRM) was operated using 30K resolution at 200 m/z, standard automated gain control (AGC) target, 350 ms maximum injection time (IT), 1 micro scan in centroid data acquisition mode. For all measurements, isolation window was 0.4, 0.7, 1 or 1.5 m/z, depending on the mass of the precursor, as recommended by the manufacturer. Ion chromatograms of all expected transitions were extracted with 12 ppm mass tolerance from the centroided spectra and deconvoluted using purpose-made R scripts. The best NCE value was selected so that the intensity of any 5th most intense transitions is maximized, thereby maximizing the chance of detecting at least 5 transitions. The retention time (RT) for each peptide was determined by LC-MS in DDA mode with an inclusion list of target peptides in all four charge-states. The MS resolution was set to 120K, 3e6 AGC target, 50 ms maximum IT and the LC operated as described below.

For LC-MS, samples were dissolved in 2.5 to 5 μl of 5% ACN in 0.1% TFA with 50 fmol of Peptide Retention Time Calibration (PRTC) Mixture (88321, Pierce™) spiked in, followed by 3 min bath sonication. All samples were analysed by liquid chromatography (U-3000, Thermo Fisher Scientific) coupled to Orbitrap Exploris 480 (Thermo Fisher Scientific). 150 fmol of BSA digest (88341, Pierce™)) and 50 fmol PRTC Mixture (free of target peptides) was systematically injected before IP samples as a negative control to test for the absence of any artefact, e.g. due to possible carryover of light contamination from SIL peptides after prior quality control tests (Salek et al. 2022). The LC gradient consisted of multiple segments. First, the solvent B (100% ACN, 0.1% FA) went from 2% to 6% and 94% A (0.1% FA in H₂O) in 5 min and then to 28.5% B in 75 min. It reached 80% in 4 min followed by 5 min wash at 80% B. Finally, the column was equilibrated for 11 min at 2% B.

For LC-MS experiments, the MS was acquired with 60K resolution at 200 m/z, over the mass range from 150 to 1450 m/z, 3e6 AGC target and 25 ms maximum IT. MS2 data was acquired with PRM scans using 60 k or 120 k resolution at 200 m/z. Target precursor list was provided with preselected charge states, the corresponding m/z and optimized collision energy values for each target, their expected retention time (+/−1 to 3 min) pre-defined with SIL peptide. The normalized AGC target was set to 1000% (or 1e6). The maximum injection time mode was set to dynamic, allowing sampling of a minimum of 5 points across the chromatographic peak. The dynamic RT feature using the Pierce PRTC mixture was active.

### Data Analysis

LC-MS data was analysed with the Skyline software v. 20.2 (MacLean et al., 2010). A minimum of top 5 intensity product ions were extracted with 7 ppm mass tolerance. Detected peaks were manually curated. Light peaks were discarded when peak retention times or shapes did not match with the heavy reference, when the normalized spectral contrast angle (NSCA) (Toprak et al., 2014) was low, or when too few transitions were detected.

The findings of the present invention are summarized also in Table 2 below; as shown, there are a vast number of peptides derivable from HPV16 E6 and E7 (approx. 9000). Prediction algorithms identify approx. ⅓ of the peptides actually binding to HLA, and wrongly predict binders in about 50% of cases. Moreover, of immunogenic peptides only ½ are correctly predicted, and of peptides actually presented on HPV16 positive tumor cells, only approx. ¼ was correctly predicted. HLA-supertype frequencies per ethnicity are shown in Table 3.

The peptides in Table 1 were found to be among the peptides evaluated with highest rates of immunoactivation in PBMC samples, inducing the strongest CD8 T cell responses, and/or being presented as HLA-complexes by the highest numbers of HPV16 positive cell lines.

TABLE 1
Preferred Peptide/HLA combinations; Protein/Position: HPV16 protein and amino acid
position the peptide is derived from, deviations from the HPV16 reference
sequence are indicated if applicable; presentation: HLA-presentation of the peptide
by HPV16-positive cells shown by MS; ELISPOT: Immungenicity of the peptide shown by
IFN-gamma ELISPOT analysis. *Highlights data from published resources

| HPV Protein/ | Amino acid | | HLA | | |
|---|---|---|---|---|---|
| Position | sequence | SEQ ID NO | restriction | ELISpot | presentation |
| E6/80-88 | ISEYRHYCY | 1 | A01 | +* | + |
| E7/11-19 | YMLDLQPET | 2 | A02 | +* | +* |
| E6/93-101 | TTLEQQYNK | 3 | A3/A11 | + | + |
| E7/49-57 | RAHYNIVTF | 4 | A24, B07, | +*(B07) | + |
| | | | B15 | +(B15) | |
| E6/15-22 | RPRKLPQL | 5 | B07 | + | + |
| E7/15-23 | LQPETTDLY | 6 | B15 | +* | + |

TABLE 2
Prediction vs. experimental confirmation of peptide functionality

| peptide type | number | peptide type | number |
|---|---|---|---|
| possible binding peptides | approx. 9000 | | |
| predicted to bind to HLA A1, | 201 | | |
| A2, A3/A11, B07, or B15 | | | |
| experientally confirmed binders | 280 | thereof predicted to bind | 99 |
| | | thereof not predicted to bind | 181 |
| experimentally confirmed | 133 | thereof not predicted | 77 |
| immunogenic peptides | | | |
| experimentally confirmed | 34 | therof not predicted | 8 |
| presented peptides | | | |

TABLE 3
HLA supertype frequencies per ethnicities (tools.iedb.org/population/, Version 3.0, Accessed 2023-02-24)

| | Ethnicity | | | | | | |
|---|---|---|---|---|---|---|---|
| Supertype | Caucasoid | Oriental | Mixed | Amerindian | African | Asian | WORLD |
| HLA-A1 | 43.2 | 14.7 | 43.2 | 6.0 | 35.0 | 28.6 | 35.0 |
| HLA-A2 | 50.7 | 46.2 | 47.1 | 77.4 | 42.2 | 38.6 | 46.9 |
| HLA-A3/A11 | 47.4 | 51.8 | 44.9 | 31.7 | 45.9 | 47.8 | 48.6 |
| HLA-A24 | 22.5 | 40.3 | 20.9 | 43.4 | 25.0 | 29.7 | 29.5 |
| HLA-B7 | 51.7 | 38.5 | 51.4 | 48.8 | 57.5 | 48.6 | 49.6 |
| HLA-B15 | 13.8 | 44.5 | 11.7 | 15.5 | 5.2 | 28.5 | 21.2 |
| Combinations: | | | | | | | |
| All 6 Supertypes | 99.9 | 99.6 | 99.5 | 100.0 | 98.9 | 98.9 | 99.7 |
| Combinations of 5: | | | | | | | |
| All without A1 | 96.3 | 98.9 | 94.8 | 99.9 | 94.9 | 96.3 | 97.1 |
| All without A2 | 95.0 | 95.7 | 94.0 | 87.9 | 93.5 | 94.8 | 95.3 |
| All without A3/A11 | 95.6 | 94.8 | 94.4 | 98.4 | 92.7 | 93.1 | 95.0 |
| All without A24 | 98.7 | 96.6 | 98.0 | 97.0 | 96.4 | 96.1 | 97.7 |
| All without B7 | 99.7 | 99.2 | 98.9 | 100.0 | 97.3 | 97.4 | 99.3 |
| All without B15 | 99.8 | 99.1 | 99.4 | 100.0 | 98.8 | 98.2 | 99.6 |
| Combinations of 4: | | | | | | | |
| All without A1, A2 | 85.7 | 93.9 | 83.8 | 86.5 | 85.8 | 90.1 | 88.9 |
| All without A1, A3/A11 | 86.8 | 92.8 | 84.6 | 97.9 | 84.7 | 87.8 | 88.5 |
| All without A1, A24 | 92.9 | 94.9 | 91.1 | 96.3 | 90.4 | 91.9 | 92.9 |
| All without A1, B7 | 91.8 | 97.8 | 88.6 | 99.8 | 87.6 | 91.5 | 93.6 |
| All without A1, B15 | 96.3 | 98.9 | 94.8 | 99.9 | 94.9 | 96.3 | 97.1 |
| All without A2, A3/A11 | 84.4 | 86.5 | 83.2 | 79.0 | 82.3 | 85.2 | 85.1 |
| All without A2, A24 | 91.1 | 89.3 | 90.0 | 74.5 | 88.5 | 89.8 | 90.2 |
| All without A2, B7 | 88.9 | 91.6 | 86.9 | 74.6 | 84.3 | 88.2 | 89.6 |
| All without A2, B15 | 95.0 | 95.7 | 94.0 | 87.9 | 93.5 | 94.8 | 95.3 |
| All without A3/A11, A24 | 91.9 | 87.9 | 90.6 | 92.0 | 87.5 | 87.5 | 89.8 |
| All without A3/A11, B7 | 90.3 | 89.8 | 87.9 | 96.7 | 82.3 | 84.4 | 88.9 |
| All without A3/A11, B15 | 95.6 | 94.8 | 94.4 | 98.4 | 92.7 | 93.1 | 95.0 |
| All without A24, B7 | 97.2 | 93.2 | 95.6 | 93.8 | 91.4 | 91.2 | 95.0 |
| All without A24, B15 | 98.7 | 96.6 | 98.0 | 97.0 | 96.4 | 96.1 | 97.7 |
| All without B7, B15 | 99.7 | 99.2 | 98.9 | 100.0 | 97.3 | 97.4 | 99.3 |

### REFERENCES

Bassani-Sternberg et al. (2016), Nat Commun, 7, 13404. doi:10.1038/ncomms13404
Becker & Riemer (2022), Frontiers Immunol, doi: 10.3389/fimmu.2022.883989
Blatnik (2018), Proteomics 18:1700390, DOI: 10.1002/pmic.201700390
Bonsack et al. (2019), Cancer Immunol Res; 7(5):719
Bourgault Villada et al., Clin Exp Immunol 2010; 159(1):45
Chardin & McCormick (1999), Cell 97, 153-155.
Chong et al. (2018), Mol Cell Proteomics, 17(3), 533-548. doi:10.1074/mcp.TIR117.000383
Currier et al. (2002), J Immunol Methods 260, 157-172
EP 0 648 503
EP 20 174 156
EP 20 196 024
EP 20 196 027
Habib et al. (2022), Cells 6; 11(3):421. doi: 10.3390/cells11030421
Hara et al., Int J Oncol 2005; 27(5):1371
Jang et al., Cancer 2012; 118(8):2173
Kast et al., J Immunol 1994; 152(8):3904
Krishna et al. (2018) Cancer Res.2018; 78(21):6159
MacLean et al. (2010), Bioinformatics, 26(7), 966-968. doi:10.1093/bioinformatics/btq054
Mizuuchi et al., Exp Mol Pathol 2012; 92(1):185
Mollenhauer et al. (1990), Biochim Biophys Acta 1031, 225-246.
Ressing et al., J Immunol 1995; 154(11):5934
Riemer et al., J Biol Chem 2010; 285(38):29608
Salek et al. (2022), Analyt Bioanalyt Chem, 414(8), 2545-2552. doi:10.1007/s00216-022-03931-w
Sidney et al. (2008), BMC Immunology 9 Art. No. 1, doi.org/10.1186/1471-2172-9-1
Stanke et al. (2010), J Immunol Methods 360, 56-65.
Steinbach & Riemer (2018), Int J Cancer 142:224
Toprak et al. (2014), Mol Cell Proteomics, 13(8), 2056-2071. doi:10.1074/mcp.0113.036475
Tsang et al. (2017), Vaccine 35(19):2605
WO 2015/162291 A1

1. A vaccine against a human papillomavirus 16 (HPV16)-related virus providing at least six discrete immunization peptides consisting of the amino acid sequences of SEQ ID NOs:1 to 6, wherein said vaccine comprises a mixture of discrete peptides each comprising exactly one of said amino acid sequences.

2. The vaccine of claim 1, wherein at least one, preferably all, of said immunization peptides are linked to at least one of a spacer, a linker, and a nanoparticle.

3. The vaccine of claim 1, wherein said nanoparticle is a silica nanoparticle with a size of at most 150 nm.

4. The vaccine of claim 1, wherein said at least one immunization peptide is linked to said nanoparticle, preferably via said spacer and/or linker, by an adsorptive bond.

5. The vaccine of claim 4, wherein said linker comprises at least one functional group being a carboxyl, carboxylate, guanidino, or amino-group.

6. The vaccine of claim 4, wherein said spacer is a self-immolative spacer.

7. The vaccine of claim 1, further comprising an anionic compound, preferably polyinosinic:polycytidylic acid, (poly(1:C)), or any derivatives thereof.

8. The vaccine of claim 1, further comprising an adjuvant, a carrier, and/or a stabilizer.

9. The vaccine of claim 1, wherein said HPV16-related virus is selected from the list consisting of HPV16, HPV31, HPV33, HPV35, HPV52, HPV58, and HPV67.

10. (canceled)

11. A method for eliciting an immune response in a human subject against HPV16-related virus infection, comprising contacting said subject with a vaccine according to claim 1.

12. The method of claim 11, wherein said eliciting an immune response is treating and/or preventing HPV16-related virus infection and/or HPV16-related inappropriate cellular proliferation.

13. The method of claim 11, wherein said subject is infected with at least one HPV16-related virus.

14. The method of claim 11, wherein the types of human leukocyte antigen (HLA) molecules produced by said subject are unknown.

15. A kit comprising the vaccine according to claim 1 comprised in a housing.
